(19) United States
(12) Patent Application Publication
(10) Pub. No.: US 2004/0122519 A1
(43) Pub. Date: Jun. 24, 2004

(54) Prosthetic glenoid

(76) Inventors: Roy C. Wiley, Warsaw, IN (US); Robert W. Zink, Warsaw, IN (US)

Correspondence Address: Jacque R. Wilson, Esq., Zimmer, Inc., 345 E. Main Street, Warsaw, IN, 46580, US

(21) Appl. No.: 10/327,433

(22) Filed: Dec. 20, 2002

(51) Int. Cl. A61F 2/30

(52) U.S. Cl. 623/18.11

(57) ABSTRACT

The invention is directed to an articulating orthopedic implant for implantation into a bone at a joint. The orthopedic implant includes a bearing component and a backing component. The bearing comprises an articulating surface for mating with a complementary implant such that rotational and translational movement is allowed therebetween. The backing of the implant includes a base, wherein the base is attached to the back of the bearing component such that the base of the bearing component is surrounded by “lip” of bearing component material.

### BACKGROUND OF THE INVENTION

1. Field of the Invention

The present invention relates generally to orthopedic joint implants such as prosthetic glenoid components. More specifically, the invention relates to prosthetic joint components comprising a bearing component and a backing component attached thereto for connection to human bone.

2. Description of the Related Art

An orthopedic implant for implantation at the shoulder joint typically includes a glenoid implant and a mating humeral implant. The glenoid implant is attached to a prepared glenoid or scapula, and the humeral implant is attached to a prepared humerus. The humeral implant usually includes a ball at an end thereof which engages and moves relative to an articulating concave surface formed in the glenoid implant. The ligaments and muscles of the body surrounding such an orthopedic implant maintain the humeral implant against the glenoid implant, while at the same time allowing relative movement therebetween.

A glenoid implant is typically formed with an articulating surface that is either fully conforming or fully non-conforming relative to the head of the humeral implant. A glenoid implant having an articulating surface that is fully non-conforming relative to the humeral head has a spherical radius that is greater than the spherical radius of the humeral head, thereby allowing rotational as well as rolling and translational movement therebetween. However, a fully non-conforming articulating surface may result in increased contact pressures between the humeral head and glenoid implant because of the relatively small surface area which is in contact therebetween at any point in time. These increased contact pressures may result in permanent deformation of the humeral head and/or articulating surface on the glenoid component over a period of time.

A fully conforming articulating surface has the same spherical radius as the humeral head, and thereby allows relative rotational movement therebetween. However, with a fully conforming articulating surface, the periphery or edge of the articulating surface is loaded by the head of the humeral implant when the arm is moved to a large degree relative to the upper body (such as when the arm is placed over the head). This edge loading at the periphery of the articulating surface may result in permanent deformation of the glenoid implant over a period of time.

Thus, a need exists for a glenoid implant that allows rotation of the humeral head relative to the articulating surface during movements of the arm relative to the upper body, while reducing the negative effect of edge loading on the articular surface of the implant.

### SUMMARY OF THE INVENTION

The present invention provides an articulating apparatus for use in a joint arthroplasty. Uses for the present invention include, but are not limited to, hip arthroplasty, knee arthroplasty, ankle arthroplasty, and shoulder arthroplasty.

The invention comprises, in one form thereof, an articular orthopaedic implant having a bearing component comprising concave articular surface and a non articular surface or “back.” The invention further comprises a backing component having a having a base attached to the back of the bearing component such that a lip of bearing component material extends around the base of the backing component, thereby providing additional material depth for the bearing component at the exterior edges thereof.

An advantage of the present invention is to reduce the negative affects of wear on the articular surface of the bearing component.

Other advantages and features of the present invention will be apparent to those skilled in the art upon a review of the following description and of the appended claims and drawings.

### BRIEF DESCRIPTION OF THE DRAWINGS

The above-mentioned and other features and objects of this invention, and the manner of obtaining them, will become more apparent and the invention itself will be better understood by reference to the following description of an embodiment of the invention taken in conjunction with the accompanying drawings, wherein:

FIG. 1 is a top perspective view of a prosthetic humeral head and a prosthetic glenoid according to the prior art.

FIG. 2 is a side view of a prosthetic humeral head and a prosthetic glenoid according to the prior art.

FIG. 3 is a side perspective view of a prosthetic glenoid having a metal backing component according to the prior art.

FIG. 4 is a side view of a prosthetic glenoid having a backing component according to the present invention.

FIG. 5 is a bottom perspective view of a prosthetic glenoid according to the present invention.

Corresponding reference characters indicate corresponding parts throughout the several views. Although the drawings represent an exemplary embodiment of the present invention, the drawings are not necessarily to scale and certain features may be exaggerated to better illustrate and explain the invention.

### DETAILED DESCRIPTION OF THE INVENTION

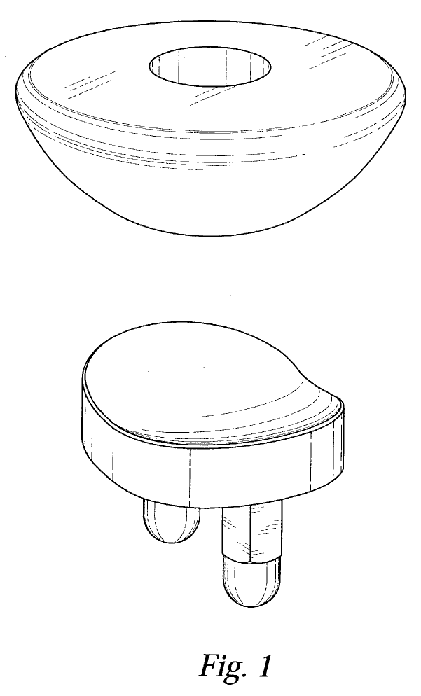

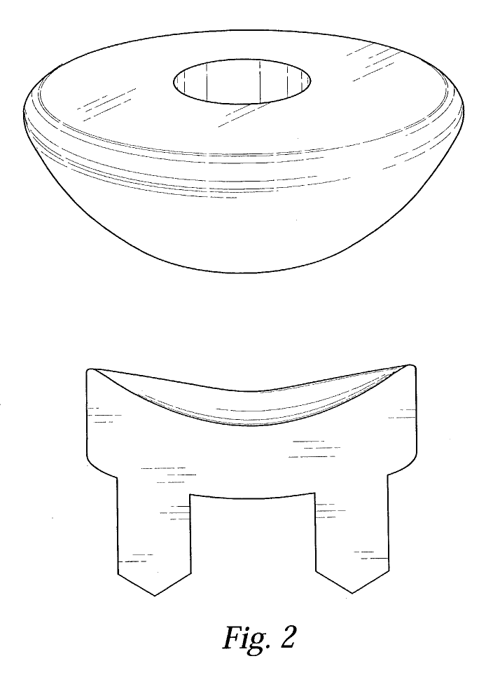

FIGS. 1, 2 and 3, show perspective and side views of a prior art prosthetic glenoid implant for implantation into the scapula of a shoulder joint and a corresponding convex humeral head suitable for articularly engaging the same. The invention is described herein with reference to a prosthetic glenoid. However, those of skill in the art will appreciate that the invention disclosed and claimed herein is equally applicable to other prosthetic devices, including for example an acetabular prosthetic device or a proximal tibia prosthetic device for use with a knee arthroplasty.

During a normal range of motion of a patients arm about his or her body at the shoulder joint, a convex prosthetic humeral head rotates and translates upon the concave articular surface of the glenoid component. In prior art implants of the type shown in FIGS. 1, 2 and 3, an edge loading condition may occur along the exterior edge of the articulating (superior) surface of the glenoid component. Such edge loading is undesirable as it tends to increase wear on the articular surface of the glenoid component. It is an object, therefore, of the present invention to reduce the effect of such edge loading by providing more material in the affected region of the glenoid implant.

Referring now to FIG. 4, there is shown a side view of a glenoid implant 100 according to the present invention, comprising a bearing component 110 and a backing component 120. Bearing component 110 comprises a material suitable for human implantation such as, cobalt, cobalt alloy, titanium, titanium alloy, stainless steel, polyethylene, ultra high molecular weight polyethylene (“UHMWPE”), cross-linked UHMWPE, or a ceramic.

Referring again to FIG. 4, bearing component 110 further comprises articular surface 112. Articular surface 112 comprises a generally concave shape suitable for mating to a convex prosthetic component, such as a humeral head. The mating component (not shown) rotationally and translationally articulates against articular surface 112. Bearing component 110 further comprises a non articular surface or back 111. Back 111 can comprise any desired shape.

Referring still to FIG. 4, glenoid implant 100 further comprises backing component 120. Backing component 120 is adapted to facilitate attaching implant 100 a bone, such as a human scapula. Backing component 120 can comprise a variety of biologically compatible materials including metals, ceramics, and plastics. However, metals, such as cobalt, cobalt alloy, titanium, titanium alloy, tantalum, or stainless steel are preferred. In one embodiment of the present invention, backing component 120 comprises a porous biocompatible metal such as porous titanium, porous tantalum, or TRABECULAR™ metal, a trademark of Implex Corporation.

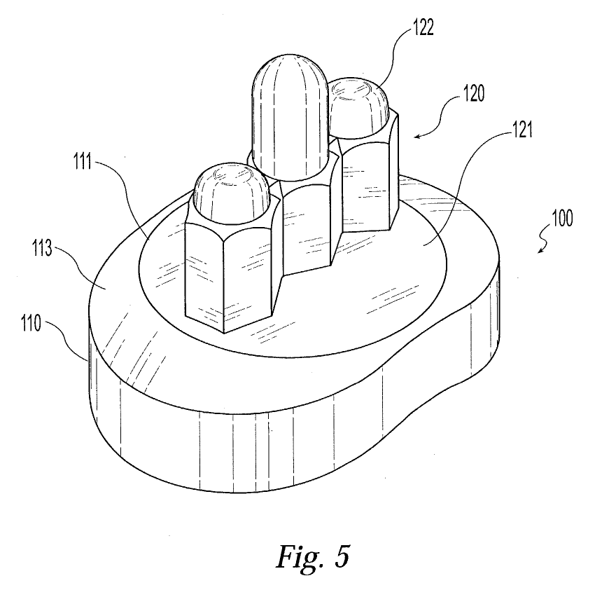

Referring now to FIG. 5, backing component 120 comprises base 121 and one or more pegs 122. In the preferred embodiment, three pegs 122 having generally cylindrical shape and convex free ends are used. Base 121 preferably comprises a generally circular shape, but could comprise any desired shape. Base 121 of backing component 120 is fixedly attached to back 111 of bearing component 110. Base 121 further comprises a smaller surface area than back 111. In the exemplary embodiment there is provided a recess on back 111 into which base 121 of backing component 120 is seated. However, those skilled in the art will appreciate that base 121 of backing component 120 can be secured to back 111 of bearing component 110 by any biologically safe means known in the art.

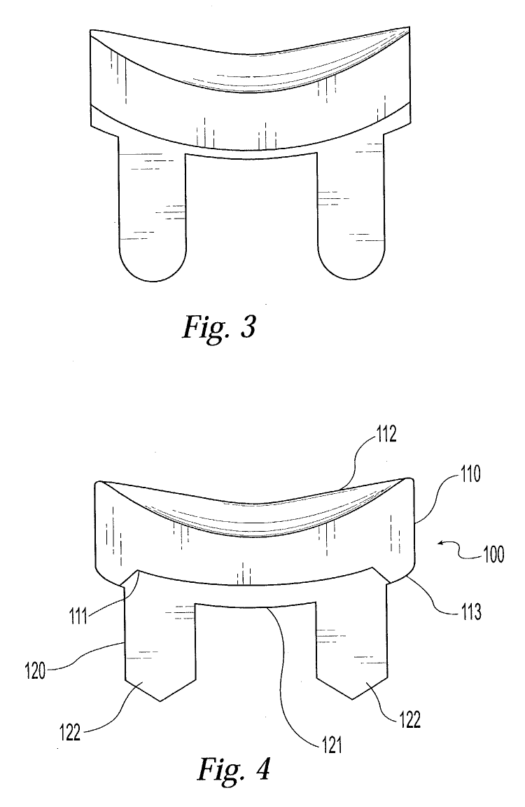

Referring again to FIG. 5, there is shown an area of back 111 of bearing component 110 extending radially from base 121 of backing component 120. This free area is called lip 113. Lip 113 denotes the area of bearing component 110 that remains unaltered by backing component 120. Lip 113 may comprise a width of about 0.1 mm to about 5 mm. These areas of bearing component 110 have greater material thickness (about 0.1 mm to about 10 mm) than prior art designs of similar overall depth wherein the backing components of prior art designs (FIG. 3) cover the entire non articular surface of the bearing component.

An advantage of the present invention is that the additional material thickness in the bearing component provides the bearing component with greater wear resistance than prior art devices without increasing the overall thickness of a part.

It will be appreciated by those skilled in the art that the foregoing is a description of a preferred embodiment of the present invention and that variations in design and construction may be made to the preferred embodiment without departing from the scope of the invention as defined by the appended claims.

We claim:

1. An articulating apparatus for use in joint arthroplasty, the apparatus comprising: a generally concave bearing component having an articular surface and a back; and a backing component having a base attached to the back of the bearing component the base having a shape and size such that a lip extends around the base.

2. The apparatus of claim 1, wherein the bearing component comprises a material selected from the group consisting of polyethylene, ultra high molecular weight polyethylene, cross linked polyethylene, cross linked ultra high molecular weight polyethylene, and ceramics.

3. The apparatus of claim 1, wherein the backing component comprises a porous material.

4. The apparatus of claim 1, wherein the backing component comprises a material selected from the group consisting of porous tantalum, porous titanium, cobalt alloy, titanium alloy, TRABECULAR metal, and ceramic.

5. The apparatus of claim 1, wherein the lip comprises a breadth of about 0.1 mm to about 5 mm.

6. The apparatus of claim 1, wherein the depth of the bearing component along its exterior edge is from about 0.1 mm to about 5 mm.

7. The apparatus of claim 1, wherein the ratio of the depth of the bearing component along its exterior edge to the depth of the bearing component at its center is from about 0.1 mm to about 10 mm.

8. The apparatus of claim 1, wherein the backing component comprises a bone fixation means comprising at least one peg extending therefrom for fixation to a bone.

9. The apparatus of claim 1, wherein the backing comprises three pegs extending therefrom for fixation to a bone.

10. The apparatus of claim 8 or 9, wherein the pegs comprise a generally cylindrical shape and a generally convex end.

11. The apparatus of claim 8, wherein the peg comprises a shape generally corresponding to the anatomy of a bone.

12. The apparatus of claim 8, wherein the peg comprises a length and shape generally suited for insertion in to the intramedullary canal of a bone.
